(19) United States
(12) Patent Application Publication
(10) Pub. No.: US 2008/0269876 A1
(43) Pub. Date: Oct. 30, 2008

(54) Repair of Incompetent Heart Valves by Papillary Muscle Bulking

(75) Inventors: Rany Huynh, Charlestown, MA (US); Theodore Lamson, Pleasanton, CA (US); Nasser Rafiee, Andover, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/837,718

(22) Filed: Aug. 13, 2007

Related U.S. Application Data
(60) Provisional application No. 60/913,710, filed on Apr. 24, 2007.

(51) Int. Cl. A61F 2/24

(52) U.S. Cl. 623/2.11

(57) ABSTRACT

Incompetency or regurgitation of a cardiac valve is treated by injecting a space occupying material or implanting a space occupying device within a papillary muscle or in heart tissue near a papillary muscle to cause lengthening or repositioning of the papillary muscle in a manner that improves coaptation of the valve leaflets and lessens valvular incompetency or regurgitation. The procedure may be performed by open thoracotomy, thoracoscopically, by a tran-endocardial catheter based approach or by a trans-coronary catheter based approach.

### RELATED APPLICATIONS

This application claims the benefit of U.S. Provisional Patent Application 60/913,710 filed Apr. 24, 2007.

### FIELD OF THE INVENTION

The present invention relates generally to medical devices and methods, and more particularly to devices and methods for using bulking agents or implantable apparatus to lengthen or otherwise adjust the position of one or more papillary muscles to improve coaptation of heart valve leaflets that are connected to such papillary muscle(s).

### BACKGROUND

The human heart includes two papillary muscles which extend as finger-like projections from the wall of the left ventricle into the left ventricular cavity. The papillary muscles are connected to leaflets of the mitral and tricuspid valves valve by way of a network of inelastic tendons known as the chordae tendineae. The papillary muscles serve, in part, to limit movement of the mitral and tricuspid valve leaflets. During the diastolic phase of the cardiac cycle, the left ventricular myocardium relaxes, thus causing the pressure within the left ventricle to decrease and causing the mitral valve leaflets to open as blood travels from the left atrium into the left ventricle. Thereafter, during the systolic phase of the cardiac cycle, the left ventricle contracts, thereby causing an increase in pressure within the left ventricle. This increase in left ventricular pressure causes the mitral valve leaflets to close. Concurrently with contraction of the left ventricle, the papillary muscles also contract causing the chordae tendineae to tighten. The tightened chordae tendineae hold the mitral valve leaflets in the proper position for closure of the valve and prevents the mitral valve leaflets from prolapsing through the valve annulus.

Mitral valve regurgitation (also known as mitral insufficiency or mitral incompetence) results when the leaflets of the mitral valve don't fully coapt (i.e., don't close tightly), thus allowing blood to backflow from the left ventricle into the left atrium during the systolic phase of the cardiac cycle. This can result in decreased cardiac output and inadequate perfusion of tissues throughout the body, with various resultant symptoms, including severe fatigue and shortness of breath.

Mitral regurgitation can result from a number of causes. In some cases, mitral regurgitation may result from shortening of one or both of the papillary muscles due to a prior myocardial infarction or cardiomyopathy. Also, in some cases, papillary muscles may shorten due to scar tissue formation in patients who have undergone a type of surgical procedure (i.e., endocardial resection) for the treatment of ventricular arrhythmias. When the papillary muscles are shortened, the chorda tendonae may create more traction on the mitral valve leaflets, preventing the leaflets from closing properly during the systolic phase of the cardiac cycle. In some cases, mitral regurgition may result from the dilation of left ventricular wall to which the papillary muscle is directly attached. In such cases, the left ventricular wall bellows out and causes the papillary muscle/chordae apparatus to be in tension, thereby preventing leaflets from fully coapting.

The prior art has included a number of surgical and interventional procedures aimed at treating mitral regurgitation by lengthening papillary muscle(s) or chordae tendineae. For example, United States Patent Application Publication No. 2006/0167474 (Bloom et al.) describes a system and method for elongating a papillary muscle by attaching a muscle elongating device to the papillary muscle.

Also, U.S. Pat. No. 6,629,534 (St. Goar, et al.) describes methods, devices, and systems for the endovascular repair of cardiac valves (particularly the atrioventricular valves and most particularly the mitral valve) wherein interventional tools, catheters and other equipment are advanced though the vasculature and to the heart chambers. The interventional tools and other equipment are then used to modify the valve leaflets, the valve annulus, the chordae tendineae and/or the papillary muscles to improve closure of the mitral valve leaflets.

Also, United States Patent Application Publication No. 2006/0287968 describes devices and methods for treatment of mitral regurgitation by deployment of implantable devices within the anterior and posterior interventricular veins, or only in the posterior interventricular vein, to cause medial displacement of the anterior and posterior interventricular veins towards the left ventricular cavity. This in turn causes repositioning of the papillary muscles in a manner that purportedly brings the mitral valve leaflets into proper coaptation during the systolic phase of the cardiac cycle.

There remains a need for the development of new devices and methods for altering the length and/or position of a papillary muscle so as to improve the function of cardiac valves to which the papillary muscle is attached.

### SUMMARY OF THE INVENTION

The present invention provides methods and systems for modifying the function of a cardiac valve by placing one or more interstitial space occupier(s) (e.g., a substance or device) within heart tissue near the valve such that the space occupier(s) will alter the shape and/or function of the valve in a manner that provides a therapeutic benefit. The interstitial space occupier(s) may be placed within myocardial tissue adjacent to the annulus of the heart valve to be treated so as not to reside within or protrude into the coronary sinus or the lumen of any coronary blood vessel, thus not obstructing or disrupting normal coronary blood flow. Also, the methods and systems of the present invention do not require attachment of any apparatus to the valve annulus or leaflets of the cardiac valve being treated.

In accordance with the present invention, there is provided a method for improving function of a cardiac valve that has at least one leaflet that is attached to a papillary muscle, such method comprising the step of implanting one or more space occupier(s) (e.g., a substance or device) in the papillary muscle or in cardiac tissue near the papillary muscle to alter the length or position of the papillary muscle in a manner that improves coaptation of the valve leaflets during closure of the valve. In some instances, the space occupier(s) may be delivered to the desired location(s) by an trans-endocardial approach wherein a catheter is introduced into the ventricle of the heart, a delivery cannula (e.g., a hollow needle) is advanced from the catheter into the papillary muscle or into the myocardium near the papillary muscle and the space occupier(s) is/are then delivered through the delivery cannula to the desired implantation site(s), thereby causing lengthening or repositioning of the papillary muscle and improved closure of the valve leaflets. In other instances, a trans-coronary approach may be used wherein a tissue penetrating catheter device is advanced into a coronary vein or coronary artery located near the intended implantation site, a delivery cannula (e.g., a hollow needle) is advanced one or more times from the tissue penetrating catheter and into the papillary muscle or into myocardial tissue near the papillary muscle and the space occupier(s) is/are then delivered through the delivery cannula to the intended implantation site(s) to cause lengthening or repositioning of the papillary muscle and a resultant improvement in closure of the valve leaflets. In some embodiments, the space occupier(s) may comprise an injectable filler substance such as collagen, hyaluronic acid, polymeric materials, hydrogels, etc. In other cases, the space occupier(s) may comprise one or more implantable device(s) such as beads, balloons or expandable members in the nature of a stent or expandable cage.

Further aspects, elements, embodiments, objects and advantages of the present invention will be appreciated by those of skill in the relevant art upon reading the detailed description and examples set forth herebelow.

### BRIEF DESCRIPTION OF THE DRAWINGS

FIG. 1 is a sectional view of a human heart having a space occupier of the present invention implanted within cardiac tissue adjacent to the antero-lateral papillary muscle for the treatment of mitral insufficiency.

FIGS. 2A-2B show steps in a trans-endocardial method for papillary bulking to treat mitral insufficiency in accordance with the present invention.

FIG. 3 is schematic illustration showing a tissue penetrating catheter system operatively inserted into a human patient and being used to perform a papillary bulking method of the present invention.

FIG. 3A is a side view of the tissue penetrating catheter device shown in FIG. 3.

FIG. 3B is an enlarged, partially fragmentary, elevational view of a distal portion of the tissue penetrating catheter device seen in FIG. 3A.

FIG. 3C is a non-fragmented cross sectional view through line 3C-3C of FIG. 3B.

FIG. 3D is a cross sectional view through line 3D-3D of FIG. 3B.

FIG. 3E is a cross sectional view through line 3E-3E of FIG. 3B.

FIG. 3F is a perspective view of the marker structure of the tissue penetrating catheter shown in FIGS. 3A-3E.

FIG. 3G is a non-fragmented cross sectional view through line 3G-3G of FIG. 3B.

FIG. 4A shows an example of an intravascular ultrasound image that the operator may see when the tissue penetrating catheter has been positioned within the coronary vasculature near the papillary muscle to be treated, but wherein the tissue penetrating catheter is not in the proper rotational orientation to cause its tissue penetrator to advance toward the intended location for implantation of the space occupier.

FIG. 4B shows an example of an intravascular ultrasound image that the operator may see when the tissue penetrating catheter has been positioned within the coronary vasculature near the cardiac valve to be treated and wherein the tissue penetrating catheter has been placed in the proper rotational orientation to cause its tissue penetrator to advance toward the intended location for implantation of the space occupier.

FIGS. 5A-5D show steps in a trans-coronary method for treatment of mitral insufficiency by implantation of a space occupying substance at the root of a papillary muscle in accordance with the present invention.

FIGS. 6A-6D show steps in another trans-coronary method for treatment of mitral insufficiency by implantation of a space occupying device at the root of a papillary muscle in accordance with the present invention.

### DETAILED DESCRIPTION AND EXAMPLES

The following detailed description, the accompanying drawings are intended to describe some, but not necessarily all, examples or embodiments of the invention. The contents of this detailed description and accompanying drawings do not limit the scope of the invention in any way.

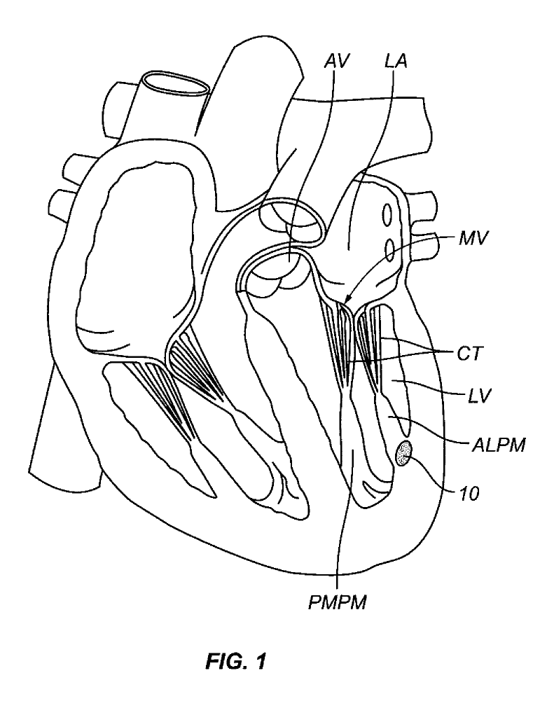

Referring to the accompanying drawings, FIG. 1 shows a sectional view of the heart of a human subject. The mitral valve MV is located between the left atrium LA and left ventrical (LV), generally adjacent to the aortic valve AV. The papillary muscles (PM) are finger-like muscular projections that extend from the wall of the left ventricle, as shown. Inelastic tendons, known as the chordae tendineae (CT) extend from the antero-lateral papillary muscle (ALPM) and from the postero-medial papillary muscle (PMPM) to the anterior and posterior leaflets of the mitral valve (MV), as shown. In this example, a space occupier 10 (e.g., a quantity of a space occupying material or a device) has been implanted within tissue at the root of the antero-lateral papillary muscle (ALPM). As explained fully herebelow, this space occupier 10 causes the antero-lateral papillary muscle ALPM to lengthen or reposition in the superior direction, thereby lessening the traction of the chordae tendonae on the anterior leaflet of the mitral valve and, thus, resulting in improved coaptation of the mitral valve leaflets during closure of the valve. It is to be appreciated that the same procedure could be performed to lengthen or reposition the posterio-medial papillary muscle (PMPM), if needed. Thus, the space occupier does not reside within, nor does it obstruct normal flow through, the coronary vasculature (e.g., it doesn't obstruct the coronary sinus, coronary veins or coronary arteries).

In some embodiments, the space occupier 10 may comprise an injectable space occupying material(s) 10a that forms a depot or mass at the interstitial location. The amount of material(s) injected will be sufficient to exert pressure on the valve annulus, thereby causing the desired shift in the position of at least one valve leaflet and resulting in improved coaptation of the valve leaflets during closure of the valve. Injectable material can be placed at the base of PM, in the PM, or around the PM in the LV wall. Examples of injectable materials that may be used for this purpose include but are not necessarily limited to; bulking agents, fat, collagens (e.g., collagens from human animal sources), crosslinked collagens (e.g., Zyplast®, Allergan-Inamed, Santa Barbara, Calif.), autologus collagen (Autologen; Collagenesis Inc., Beverly, Mass.); polymethylmethacrylate microspheres suspended in bovine collagen (Artecoll®; Rofil Medical International NV, Breda, The Netherlands), acellular freeze dried human cadaveric dermis (AlloDerm®, LifeCell Corporation, Branchburg, N.J.), micronized acellular freeze dried human cadaveric dermis (Cymetra®, LifeCell Corporation, Branchburg, N.J.), cultured autologous fibroblasts (Isolagen®, Isolagen Technologies, Inc., Exton, Pa.), hyaluronic acid, crosslinked hyaluronic acid (Hylaform® gel; Allergan-Inamed, Santa Barbara, Calif.; and Genzyme Corporation, Cambridge, Mass.), stabilized hyaluronic acid derivatives (Restylane®, Q-Med AB, Uppsala, Sweden), calcium hydroxyl appetite suspension (Radiesse®, Bioform Medical, Inc., San Mateo, Calif.), solubilized elastin peptides with bovine collagen (Endoplast-50®, Laboratoiries Filorga, Paris, France), dextran beads suspended in hylan gel (Reviderm®, Rofil Medical International NV, Breda, The Netherlands), silicones (e.g., high-viscosity liquid silicone such as Adatosil-5000™ and Silikon-1000™, Dow Corning, Midland Mich.), poly-L-lactic acid (Sculptra®, Dermik Aesthetics, Berwyn, Pa.), expanded polytetrafluoroethylene (e-PTFE) (e.g., SoftForm™ from Collagen Aesthetics, Inc., acquired by Allergan-Inamed, Santa Barbara, Calif. or Advanta™ from Atrium Medical Corporation, Hudson, N.H.), etc.

In other embodiments, the space occupier 10 may comprise one or more implantable space occupying device(s) 10b. Such implantable space occupying device(s) 10b may comprise one or more relatively simple space occupying articles or apparatus such as, for example, beads, balls, filament(s), strand(s), coils, suture material, etc. Or, such implantable device(s) may comprise and expandable implant such as a stent, an expandable cage, expandable cylinder, expandable ball, other expandable structures, implantable balloons, implantable balloons filled with solid or gellatenous material and implantable, tissue expanders, etc.

During injection of the space occupying material 10a or during implantation and/or expansion of the space occupying device 10b, the operator may use echocardiography or any other suitable means to observe the movement of the valve leaflets continually in real time, or at selected intervals, to determine when the papillary muscle(s) has/have been lengthened or repositioned sufficiently to provide a desired improvement in closure of the valve during the phase of the cardiac cycle when that particular valve should close (e.g., during systole in the case of a mitral valve).

In some applications of the invention, the injectable material or device comprising the space occupier 10 may be injected or introduced into the desired interstitial location during an open-chest surgical procedure or using minimally invasive thoracoscopic techniques known in the art. In other applications, the injectable material or device comprising the space occupier 10 may be delivered by catheter(s) using either a trans-endocardial or trans-coronary approach, examples of which are described fully herebelow.

### Trans-Endocardial Delivery of the Space Occupier

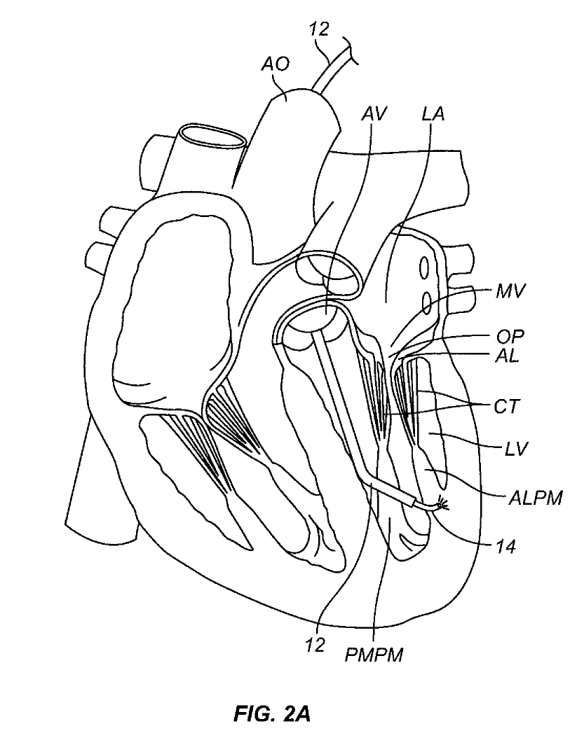

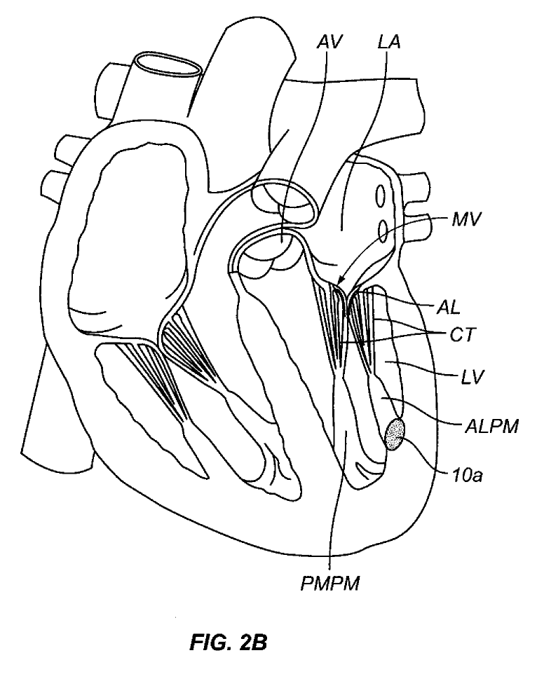

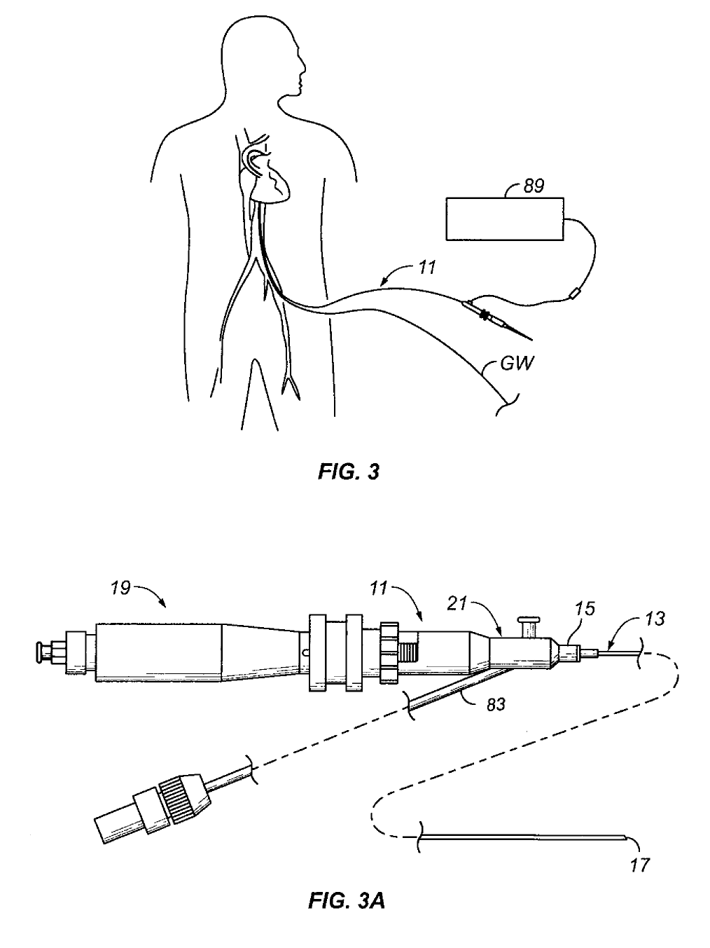

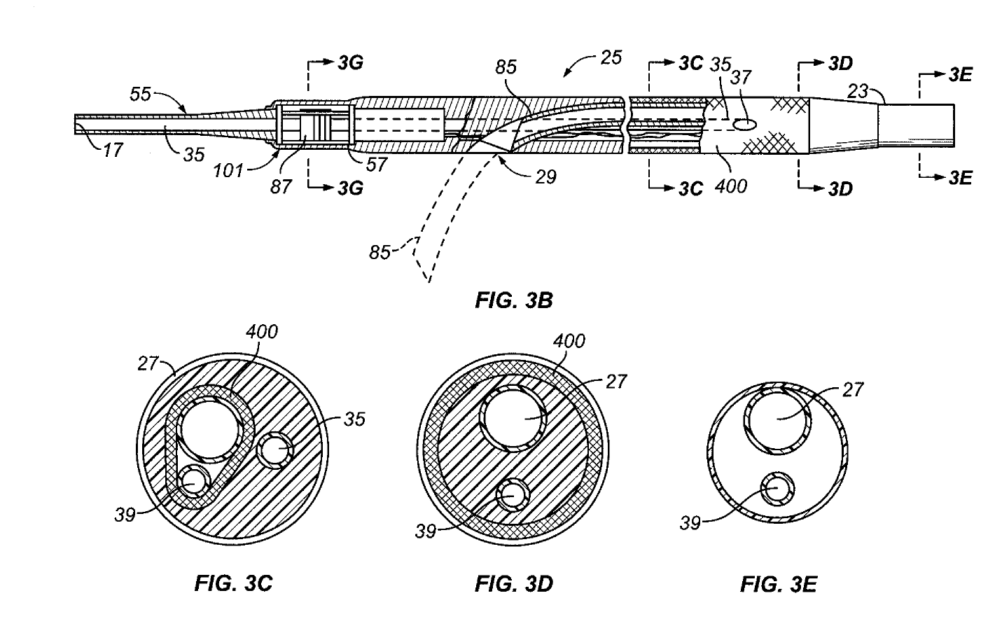

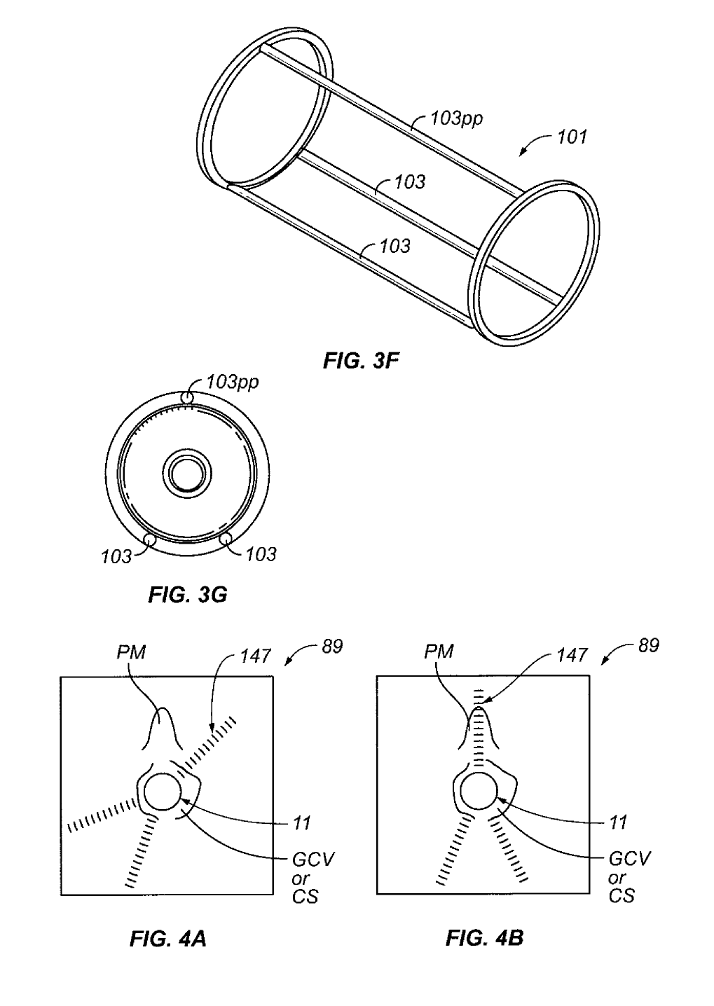

FIGS. 2A-2B show a trans-endocardial method for treatment of mitral insufficiency in accordance with the present invention. As seen in FIG. 2A, in this example, the leaflets of the mitral valve MV are not in coaptation and an opening OP exists between the mitral valve leaflets during the systolic phase of the cardiac cycle. To improve coaptation of the mitral valve leaflets, a catheter 12, such as a steerable or non-steerable guide catheter is inserted into the arterial vasculature and is advanced in retrograde fashion through the aorta AO, through the aortic valve AV and into the left ventricle LV. The distal end of the catheter 12 is positioned such that it is directed at the root of the antero-lateral papillary muscle ALPM. Techniques known in the art of medical imaging and/or interventional cardiology and radiology may be used to facilitate positioning of the catheter 12. For example, flouroscopy (traditional bi-plane or O-arm) as well as ultrasound (2D, 3D or 4D) can be use to position catheter 12. Also, other ventricular mapping systems like three dimensional computed tomography (CT) mapping (e.g., using the CARTO™ mapping systems available from Biosense-Webster, Inc., Diamond Bar, Calif.), other CT scans or MRI scans can be used to map the ventricle to facilitate the desired positioning of the catheter 12. Thereafter, a delivery cannula 14 (e.g., a hollow needle) is advanced out of the distal end of the catheter 12 and into the myocardium at the root of the antero-lateral papillary muscle ALPM. Alternatively, a single catheter having a hollow needle or injector advanceable thereform may be used in this application. One example of such a catheter is shown in FIGS. 3-3G and described herebelow (in relation to a trans-coronary approach for this papillary muscle bulking procedure) and is commercially available as the Pioneer Catheter (Medtronic Vascular, Inc., Santa Rosa, Calif.).

A space occupying substance or material is then injected through the cannula 14 forming a depot of space occupying material 10 within tissue at the root of the antero-lateral papillary muscle ALPM. The operator may use echocardiography, contrast angiography or other techniques to monitor the coaptation of the mitral valve leaflets and/or regurgitation through the valve, so that injection of the substance may continue until a desired level of improvement is seen in the coaptation of the valve leaflets. As seen in FIG. 2B, the catheter 12 and cannula 14 are then removed. The implanted space occupying material 10a causes lengthening of the antero-lateral papillary muscle ALPM resulting in improved coaptation of the valve leaflets and closure of the opening OP when the mitral valve is in its closed position. The implanted space occupying material can also bulk the left ventricular wall at or near the location of the papillary muscle PM thereby relieving the tension on the chordae tendineae (CT).

Also, in some applications of the invention, it may be desired to deliver a space occupying substance that is formed by mixing two or more component substances. In such applications, an injector device having 2 or more lumens may be used to inject the component substances so that they become combined in situ at the implantation site or within the injection device shortly before the resultant component mixture enters the implantation site. Examples of multiple-component injector devices that may be used for injection of multiple components in this manner include but are not necessarily limited to those described in copending U.S. Provisional Patent Application No. 60/878,527 filed Jan. 3, 2007 and is a continuation in part of U.S. patent application Ser. No. 11/426,219 filed Jun. 23, 2006 (published as United States Published Patent Application 2007-0014784), which claims priority to U.S. Provisional Patent Application Nos. 60/693,749 filed Jun. 23, 2005 and 60/743,686 filed Mar. 23, 2006, the entire disclosure of each such application being expressly incorporated herein by reference.

### Trans-Coronary Delivery of the Space Occupier

FIGS. 3A-3G show a tissue penetrating catheter 11 that may be used for trans-coronary delivery of the space occupier 10. This tissue penetrating catheter 11 includes an elongated catheter body 13 having a proximal end 15, a distal end 17, a handle 19 and a hub 21 coupled to the proximal end of the catheter body 15 and to the handle. The handle 19 may also serve as a controller for use in advancing and retracting the penetrating instrument, such as a tissue penetrator 85 described more fully below.

### The Catheter Body

The catheter body 13 includes a relatively rigid proximal section 23 which may be constructed, for example, of a metal hypo tube and an elongated flexible distal section or region suitably joined to and extending distally from the proximal section. A hand piece 19 is attached to the proximal end of the proximal section 23, as shown. In the preferred embodiment the hand piece 19 and proximal section 23 are approximately 100 cm in length. The flexible distal section may incorporate a reinforcement member such as a wire braid 400 as shown in FIG. 3D and, which in the example shown may be approximately 30 cm in length. This braid 400 may terminate approximately 3 cm from the distal end 17.

In this example, the catheter body 13 has a penetrator lumen 27 that terminates distally at an exit location or exit port 29 on the side wall of the catheter. The penetrator lumen 27 extends proximally from the exit port 29 to the proximal end 15 of the catheter body 13 and communicates with the interior of the handle 19 through the hub 21. The penetrator lumen 27 contains the tissue penetrator 85, which is advanceable from the catheter body 13 through the wall of the coronary sinus or coronary blood vessel in which the catheter body 13 is positioned and to an interstitial location within heart tissue. The exit port 29 is preferably located a short distance proximal to the distal tip 17. A radiopaque marker may be mounted on the lumen 27 adjacent the exit port 29.

In some applications, the space occupying substance may be formed by mixing two or more component substances. In such applications, the penetrator 85 or other injector device may have 2 or more lumens may be used to inject the component substances so that they become combined in situ at the implantation site or within the injection device shortly before the resultant component mixture enters the implantation site. Examples of other multiple-component injector devices that may be used for injection of multiple components in this manner include but are not necessarily limited to those described in U.S. Provisional Patent Application No. 60/878,527 filed Jan. 3, 2007 and in U.S. patent application Ser. No. 11/426,219 filed Jun. 23, 2006 (published as United States Published Patent Application 2007-0014784), which claims priority to U.S. Provisional Patent Application Nos. 60/693,749 filed Jun. 23, 2005 and 60/743,686 filed Mar. 23, 2006, the entire disclosure of each such application being expressly incorporated herein by reference.

The catheter body 13 may also have a guidewire lumen 35 which extends to the distal end 17 of the catheter body 15. In this embodiment, the guidewire lumen 35 extends proximally to an inlet port 37 on the catheter side wall adjacent to the proximal section 23. The catheter body also has a lead lumen 39 for a purpose described below.

In this example, the catheter includes a tapered distal tip section 55 of soft, flexible, biocompatable material and exit port 29 is spaced slightly proximally of shoulder 57.

### Imaging Transducer

An imaging transducer 81 is mounted on the distal tip section 55 just distal to shoulder 57. In this embodiment, the imaging transducer 81 comprises a phased array transducer (e.g, an intravascular ultrasound transducer or IVUS) operative to image 360° about the catheter 11. This imaging transducer 87 comprises an annular array of individual crystals or elements coupled to a multiplex circuit which is within the major section 51 of the catheter body 13 adjacent the shoulder 57. The multiplex circuit is in turn coupled to leads which extend through the lead lumen 39 and a port or sidearm 83 of the hub 21 to an imaging console. When activated, the imaging transducer 87 emits ultrasound signals and receives back echos or reflections which are representative of the nature of the surrounding environment. The imaging transducer 81 provides an imaging signal from which an image of the surrounding structure can be created by signal processing apparatus located in the imaging console and viewed on a standard display screen. A suitable phased array transducer, the accompanying circuitry and the imaging console may be obtained commercially from Endosonics of Rancho Cordova, Calif. or Intravascular Research Limited (United Kingdom).

### Orientation Marker

An imageable marker structure 101 is fixedly mounted on the catheter body 13 in a known circumferential orientation relative to the exit port 29. As seen in FIG. 3F, this marker structure 101 is generally in the form cage having three longitudinal members 103 and 103pp. As seen in FIG. 3B, this marker structure 101 is mounted on the catheter such that the transducer 81 is within the longitudinal members 103 and 103pp. The longitudinal members 103 and 103pp are disposed at circumferentially spaced apart locations. Each of these longitudinal members creates a bloom or echo on the ultrasound image, as illustrated in FIGS. 4A and 4B. One of the longitudinal members 103pp is positioned at a circumferential position that is axially aligned with the exit port 29 or otherwise positioned to be indicative of the trajectory on which the tissue penetrator 85 will advance from the catheter body 13 and is designated as the penetrator path indicating member 103pp. As seen on FIGS. 4A and 4B and described more fully herebelow, this penetrator path indicating member 103PP provides a penetrator path indication 147 on the image display, thereby showing the operator a projection of the trajectory that will be followed by the tissue penetrator when the tissue penetrator 85 is subsequently advanced from the catheter body 13.

FIGS. 4A and 4B are an illustration of what the operator may see on the display screen of the imaging console 89 during performance of a trans-coronary method of the present invention using the particular tissue penetrating catheter 11 shown in FIGS. 3A-3G. Specifically, in FIG. 4A, the tissue penetrating catheter 11 has been inserted and advanced to a position within a coronary blood vessel that is close to the antero-lateral papillary muscle ALPM (e.g., the posterior interventricular vein or posterior interventricular artery). On the image displayed from the imaging transducer 87, one can see the surrounding wall of the coronary blood vessel in which the catheter 11 is positioned as well as an image of the antero-lateral papillary muscle ALPM. The penetrator trajectory image 147 created by the penetrator path indicating longitudinal member 103pp is visually distinguishable from the images created by the other longitudinal members 103 of the marker structure 101. In the example of FIG. 4A, this penetrator trajectory image 147 is not directed toward the antero-lateral papillary muscle ALPM, but rather to one side of the antero-lateral papillary muscle ALPM. This indicates that, if the tissue penetrator 85 were to be advanced from the catheter body 13 without first adjusting the rotational orientation of the catheter 11, the penetrator 85 would not travel in the direction of the antero-lateral papillary muscle ALPM, as desired. In view of this, the operator may rotate the catheter 11 until the penetrator trajectory image 147 is directed at the antero-lateral papillary muscle ALPM or otherwise toward the location to which it is intended for the penetrator 85 to advance.

It will be appreciated that, as an alternative to the use of the marker structure 101, the imaging transducer 87 could be mounted in a fixed position and a selected one (or selected ones) of the individual imaging elements (e.g., crystals) of the phased array may be selected as being in longitudinal alignment with the outlet aperture 29 or otherwise located so as to be indicative of the trajectory on which the penetrator 85 will advance from the catheter body 13. This selected imaging element(s) 121 shall be referred to herein as the Apenetrator-path-indicating imaging element(s).” The imaging console 86 may include a computer or processor that is programed to display on the imaging display a marking (e.g., a vertical line or other suitable making) that is in aligned with the radial location of the penetrator-path-indicating imaging element(s). Thus, such marking will serve as a visual indicator of the trajectory that will be followed by the tissue penetrator 85 as it is advanced from the catheter body 13. It will be appreciated by those of skill in the art that this marking may be created on the imaging display screen electronically (e.g., as an illuminated or colored line on the image) or it may be physically marked on the screen (e.g., by felt tipped marker or other suitable marking material or apparatus such as a template). In such embodiments, the operator may rotate the catheter until the marking (e.g., vertical line) passes directly through the image of the cardiac valve to be repaired, thus indicating to the operator that when the tissue penetrator 85 is subsequently advanced from the exit port 29, it will advance toward the intended implantation site in a papillary muscle or within the myocardium near a papillary muscle, and not in some other radial direction.

Also, as an alternative to the use of the marking 101 and any on-board imaging transducer 81, the catheter may include suitable radiographic marking to allow the operator to rotationally adjust and radially orient the catheter using fluoroscopy or other radiographic imaging.

### Method for Trans-Coronary Delivery of Space Occupying Substance

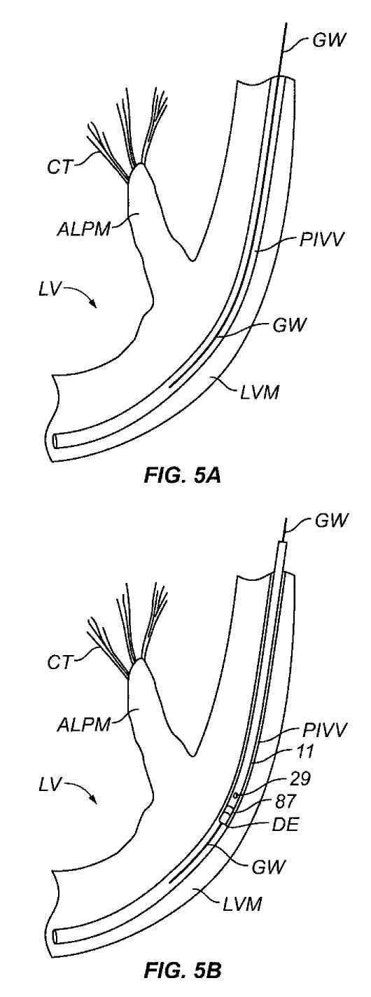

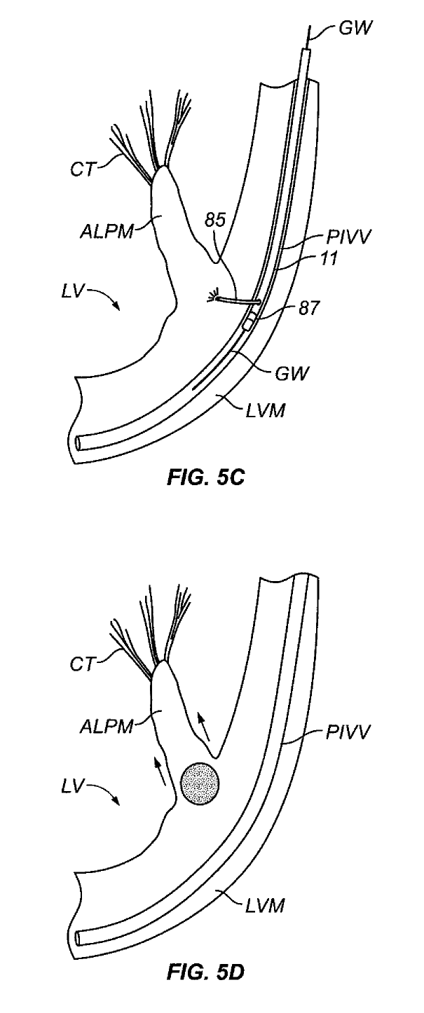

FIGS. 5A through 5D show steps in a method wherein the above described tissue penetrating catheter device 11 is used to inject a space occupying material into tissue at the root of the antero-lateral papillary muscle ALPM to cause that papillary muscle to become longer or to displace in the superior direction thereby improving the closure of the mitral valve leaflets and lessening regurgitation through the mitral valve MV.

As seen in FIG. 5A, a guidewire GW is initially advanced into the posterior interventricular vein PIVV past the root of the antero-lateral papillary muscle ALPM. Those of skill in the art will appreciate that other coronary vessels, such as the posterior interventricular artery, may be used as an alternative to the posterior interventricular vein PIVV.

Thereafter, as shown in FIG. 5B, the tissue penetrating catheter 11 (with its tissue penetrator 85 in the retracted position) is advanced over the guidewire GW to a position where the tissue penetrator outlet port 29 is near the root of the adjacent antero-lateral papillary muscle ALPM. If the catheter 11 is equipped with the optional imaging transducer 87, the imaging transducer will then be actuated and the operator, while viewing an image from the imaging transducer 87, will rotate the catheter 11 as needed until the penetrator path indication 147 is aligned with the location where it is intended to inject the space occupying material, such as the root of the papillary muscle.

After the catheter 11 has been positioned and rotationally oriented so that the penetrator 85 is effectively aimed at the desired location, the penetrator 85 is advanced to the desired location and the space occupying material is injected through the lumen of the penetrator 85, as seen in FIG. 5C. The advancement and positioning of the penetrator 85 may be monitored or verified using the optional imaging transducer 87 of the catheter 11 and/or other suitable means such as by fluoroscopy.

As seen in FIG. 5D, after the space occupying material 10a has been injected, the penetrator 85 is retracted into the catheter 11 and the catheter 11 and guidewire GW are removed. The implanted space occupying material 10a exerts pressure on the antero-lateral papillary muscle ALPM causing it to lengthen or otherwise extend in the superior direction. This allows the anterior leaflet of the mitral valve to resume a more normal position and facilitates coaptation of the mitral valve leaflets during closure of the valve. In this manner, mitral regurgitation will be eliminated or improved. During injection of the space occupying material, the positioning of the mitral valve leaflets may be monitored by echocardiography and/or the competency of the valve may be monitored by dye contrast angiography or other suitable means to determine when the amount of the space occupying material 10a injected has been adequate to bring about the desired improvement in leaflet coaptation or valve function.

It will be appreciated that this same procedure could be performed to lengthen or cause repositioning of the postero-medial papillary muscle PMPM or for other valves such as the tricuspid valve.

### Method for Trans-Coronary Delivery of Space Occupying Device

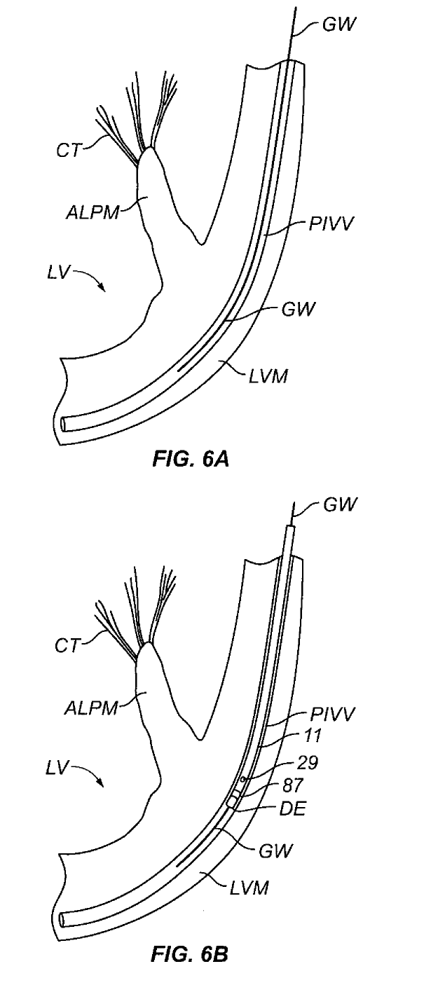

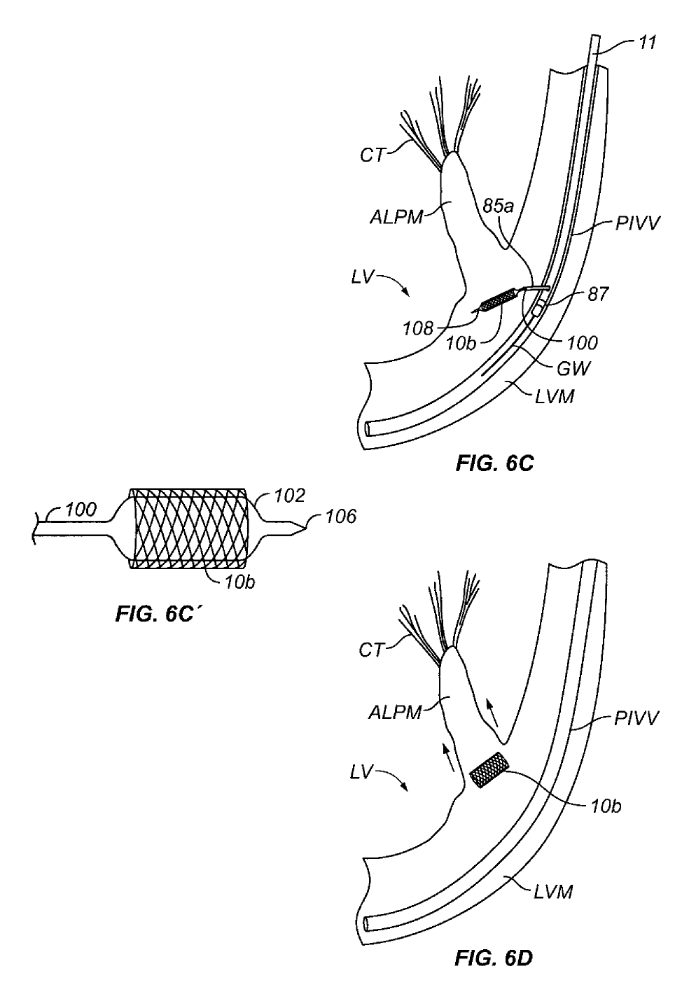

FIGS. 6A through 6D show steps in a method wherein the above described tissue penetrating catheter device 11 is used to implant a space occupying device 10b within tissue at or near the root of the antero-lateral papillary muscle ALPM for the purpose of improving closure of the mitral valve leaflets and lessening regurgitation through the mitral valve MV.

As seen in FIG. 6A, a guidewire GW is initially advanced into the posterior interventricular vein PIVV past the root of the antero-lateral papillary muscle ALPM. Those of skill in the art will appreciate that other coronary vessels, such as the posterior interventricular artery, may be used as an alternative to the posterior interventricular vein PIVV.

Thereafter, as shown in FIG. 6B, the tissue penetrating catheter 11 (with its tissue penetrator 85 in the retracted position) is advanced over the guidewire GW to a position where the tissue penetrator outlet port 29 is near the root of the adjacent antero-lateral papillary muscle ALPM. If the catheter 11 is equipped with the optional imaging transducer 87, the imaging transducer will then be actuated and the operator, while viewing an image from the imaging transducer 87, will rotate the catheter 11 as needed until the penetrator path indication 147 is aligned with the location where it is intended to inject the space occupying material, such as the root of the papillary muscle.

As seen in FIG. 6C and 6C′, after the catheter 11 has been positioned and rotationally oriented so that the penetrator 85 is effectively aimed at the desired location, the penetrator 85 is advanced to the desired location and a device delivery catheter 100 having the space occupying device 10b mounted thereon, is advanced out of the penetrator 85 at the location where it is desired to implant the space occupying device 10b. The advancement and positioning of the penetrator 85 and/or delivery catheter 100 may be monitored or verified using the optional imaging transducer 87 of the catheter 11 and/or other suitable means such as by fluoroscopy.

Examples of small balloon-expandable stents that may be used as the space occupying device 10b and delivery catheters therefore include the Guidant MULTI-LINK RX PIXEL® Coronary Stent System (Abbott Vascular, Inc., Santa Clara, Calif.) and the Micro-Driver® Coronary Stent System (Medtronic Vascular, Inc., Santa Rosa, Calif.). Another small balloon catheter device that may be used for delivery and expansion of the space occupying device 10b, such as a balloon-expandable stent, is an occlusion wire having an occlusion balloon with a deflated diameter of about 0.028 inch and a fully inflated diameter of about 5.5 mm (GuardWire® Temporary Occlusion System, Medtronic Vascular, Inc., Santa Rosa, Calif.). The balloon catheter 100 or other delivery catheter used to deliver the space occupying device 10b may in some embodiments have a sharp distal tip 106 to facilitate its desired advancement through tissue.

In the particular example shown in FIG. 6C′, the device delivery catheter 100 comprises a balloon catheter having a balloon 102 on which a radially expandable space occupying device 10b is mounted. The balloon catheter 100 may optionally have a sharp distal tip 106 to facilitate its desired advancement through tissue. When the device 10b has been positioned at its intended implantation site, the balloon 102 is inflated, thereby causing the device 10b to expand, exerting force on the antero-lateral papillary muscle ALPM. In the particular example described here, the space occupying device 10b comprises a plastically deformable device that plastically deforms to its expanded configuration as the balloon 102 is inflated. It is to be appreciated, however, that in other embodiments of the invention, the space occupying device 10b may be self-expanding and the delivery catheter 100 may have apparatus (e.g., a sheach or clip) for constraining such self expanding device until the constraining apparatus has been removed allowing the device to self expand.

The implanted space occupying device 10b exerts pressure on the antero-lateral papillary muscle ALPM causing it to lengthen or otherwise extend in the superior direction. This allows the anterior leaflet of the mitral valve to resume a more normal position and facilitates coaptation of the mitral valve leaflets during closure of the valve. In this manner, mitral regurgitation will be eliminated or improved. During expansion of the space occupying device 10b, the positioning of the mitral valve leaflets may be monitored by echocardiography and/or the competency of the valve may be monitored by dye contrast angiography or other suitable means to determine when the device 10b has been expanded to an extent that is adequate to bring about the desired improvement in leaflet coaptation or valve function.

As shown in FIG. 6D, after the device 10b has been expanded, the balloon 102 is deflated, the balloon catheter 100 and penetration member 85 are retracted into the catheter 11 and the catheter 11 and guidewire GW are removed, leaving just the expanded device 10b in place.

It will be appreciated that this same procedure could be performed to lengthen or cause repositioning of the postero-medial papillary muscle PMPM.

It is to be further appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless to do so would render the embodiment or example unsuitable for its intended use. Also, where the steps of a method or process are described, listed or claimed in a particular order, such steps may be performed in any other order unless to do so would render the embodiment or example not novel, obvious to a person of ordinary skill in the relevant art or unsuitable for its intended use. All reasonable additions, deletions, modifications and alterations are to be considered equivalents of the described examples and embodiments and are to be included within the scope of the following claims.

What is claimed is:

1. A method for improving function of a cardiac valve that has at least one leaflet that is attached to a papillary muscle, said method comprising the step of:
A) implanting a space occupier in the papillary muscle or in cardiac tissue near the papillary muscle to alter the length or position of the papillary muscle in a manner that improves coaptation of the valve leaflets during closure of the valve.

2. A method according to claim 1 wherein the space occupier comprises a quantity of space occupying substance and wherein Step A comprises delivering said space occupying substance to the interstitial location within heart tissue.

3. A method according to claim 2 wherein the space occupying material is injectable through the lumen of an delivery cannula and wherein Step A comprises:
i) inserting an delivery cannula into the heart; and
ii) injecting the space occupying material through the delivery cannula to form a depot of the space occupying material within a papillary muscle or within heart tissue near a papillary muscle.

4. A method according to claim 3 wherein the delivery cannula comprises a needle having one or more lumens.

5. A method according to claim 3 wherein the delivery cannula is advanceable from a tissue penetrating catheter and wherein the step of inserting the delivery cannula into the heart comprises:
inserting the tissue penetrating catheter into the subject's vasculature;
advancing the tissue penetrating catheter through the subject's vasculature to a location within the coronary vasculature;
advancing the delivery cannula from the tissue penetrating catheter and into cardiac tissue; and
injecting the space occupying material through the delivery cannula to form a depot of the space occupying within a papillary muscle or within heart tissue near a papillary muscle.

6. A method according to claim 5 wherein the delivery cannula is part of the tissue penetrating catheter.

7. A method according to claim 6 wherein the tissue penetrating catheter has a tissue penetrator that has a lumen and an open distal end, said tissue penetrator being advanceable from the tissue penetrating catheter into the cardiac tissue, and wherein:
the step of advancing the delivery cannula from the tissue penetrating catheter and into cardiac tissue comprises;
i) advancing the tissue penetrator from the tissue penetrating catheter into cardiac tissue; and
ii) advancing the delivery cannula through the lumen of the tissue penetrator and out of its open distal end.

8. A method according claim 7 wherein the delivery cannula comprises a flexible catheter.

9. A method according to claim 8 wherein the delivery cannula has a tissue penetrating distal end so that it may penetrate further through cardiac tissue after exiting the distal end opening of the tissue penetrator.

10. A method according to claim 5 wherein the tissue penetrating catheter is equipped with orientation apparatus useable to determine the trajectory on which the delivery cannula will advance and wherein the method further comprises the steps of:
using the orientation apparatus to determine a projected trajectory on which the delivery cannula will advance; and
adjusting the rotational orientation of the catheter as needed so that the projected trajectory on which the delivery cannula will advance is in the direction of the intended implantation location.

11. A method according to claim 7 wherein the tissue penetrating catheter is equipped with orientation apparatus useable to determine the trajectory on which the tissue penetrator will advance and wherein the method further comprises the steps of:
using the orientation apparatus to determine a projected trajectory on which the tissue penetrator will advance; and
adjusting the rotational orientation of the catheter as needed so that the projected trajectory on which the tissue penetrator will advance is in the direction of the intended implantation location.

12. A method according to claim 5 wherein the tissue penetrating catheter is inserted into the subject's venous vasculature and advanced into the coronary sinus or a coronary vein of the subject's heart.

13. A method according to claim 5 wherein the tissue penetrating catheter is inserted into the subject's arterial vasculature and advanced into a coronary artery.

14. A method according to claim 2 wherein the space occupying substance is selected from the group consisting of:
collagens;
hyaluronic acid;
polymeric materials; and
hydrogels.

15. A method according to claim 2 wherein the space occupying substance expands after it has been delivered to the interstitial location within heart tissue.

16. A method according to claim 1 wherein the space occupier comprises at least one space occupying device.

17. A method according to claim 16 wherein the space occupying device is selected from the group consisting of beads, balls, filaments, stents, cages, expandable structures, implantable balloons, implantable balloons filled with solid or gellatenous material and implantable tissue expanders.

18. A method according to claim 16 wherein the space occupying device is expandable from a non-expanded configuration to an expanded configuration and wherein the method comprises:
i) advancing the space occupying device to the interstitial location within heart tissue while in its non-expanded configuration; and
ii) causing the space occupying device to expand to its expanded configuration.

19. A method according to claim 18 wherein the space occupying device self-expands and wherein constraint is applied to the space occupying device so that it is constrained in a non-expanded configuration while being delivered to the implantation location and, thereafter, the constraint is removed to allow the space occupying device to self-expand to an expanded configuration.

20. A method according to claim 18 wherein the space occupying device is plastically deformable to its expanded configuration and wherein the space occupying device is delivered to the interstitial location within heart tissue while in its non-expanded configuration and is thereafter plastically deformed to its expanded configuration.

21. A method according to claim 20 wherein the space occupying device is delivered by a delivery catheter that has a balloon, and wherein, during delivery of the space occupying device to the implantation location, the balloon is deflated and the space occupying device is mounted on the deflated balloon and, thereafter, the balloon is inflated thereby causing the space occupying device to expand to its non-expanded configuration.
